(19) United States
(12) Patent Application Publication
(10) Pub. No.: US 2012/0157853 A1
(43) Pub. Date: Jun. 21, 2012

(54) Acoustic Transducer Incorporating an Electromagnetic Interference Shielding as Part of Matching Layers

(75) Inventors: Jean-Francois Gelly, Sophia Antipolis (FR); Frederic Lanteri, Sophia Antipolis (FR)

(73) Assignee: GENERAL ELECTRIC COMPANY, Schenectady, NY (US)

(21) Appl. No.: 12/968,961

(22) Filed: Dec. 15, 2010

(51) Int. Cl. A61B 8/00

(52) U.S. Cl. 600/459

(57) ABSTRACT

Ultrasound probes, and methods of forming probes, with electromagnetic shielding and/or improved heat management are provided. Certain probes include an acoustic stack including an active layer, a protection face plate or lens, and a matching layer. The matching layer includes a mass layer and a spring layer. The probe further includes a cable configured to communicate signals to and from the ultrasound probe. The probe further includes an electromagnetic radiation shield comprising the mass layer. The shield encompasses the active layer and the cable, and is grounded via an electrode. The shield is configured to inhibit external electromagnetic radiation from interfering with the signals communicated to and from the ultrasound probe via the cable. Certain probes are configured such that the mass layer is thermally connected to a thermal drain or a heat sink such that heat is conducted away from the protection face plate or lens.

### RELATED APPLICATIONS

U.S. application Ser. No. 12/406,731 entitled “MULTI-LAYERED IMPEDANCE MATCHING STRUCTURE FOR ULTRASOUND PROBE”, which application was published Sep. 23, 2010 as U.S. Application Publication No. 2010/0237746, is incorporated by reference herein in its entirety.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

Embodiments of the present technology generally relate to ultrasound probes, and more particularly, to electromagnetic shields for ultrasound probes.

An ultrasound probe typically has many acoustical stacks that each correspond to an imaging element of the probe. Each acoustical stack has several layers that are attached together in a stacked configuration. The active stack comprises at least one electro-mechanical structure within the stack, for instance a piezoelectric layer, for instance formed of a piezoelectric material, such as piezoelectric ceramic, single crystal or a piezocomposite material, that has high impedance compared to the acoustic impedance of water or human tissues. Matching layers are provided on the top side of the active layer to transform the acoustic impedances between the active layer that has high impedance and an exterior or protection face plate or lens protection face plate (often also used as a protection face plate or lens) of the probe that has low impedance. The low impedance may be based on the acoustic impedance of water, a human, or other subject to be scanned. Acoustic stack configurations for ultrasound probes are described, for example, in U.S. application Ser. No. 12/406,731 entitled “MULTI-LAYERED IMPEDANCE MATCHING STRUCTURE FOR ULTRASOUND PROBE”, which application was published Sep. 23, 2010 as U.S. Application Publication No. 2010/0237746.

Ultrasound transducers can experience electromagnetic interference due to surrounding equipment. One way to protect ultrasound transducers from such electromagnetic interference is to incorporate a metal layer between the outer matching layer and the protection face plate or lens. However, such a solution can degrade transducer bandwidth and pulse shape due to the acoustic impedance of the metal shield being much higher than the acoustic impedance of the outer matching layer.

Thus, it is desirable to provide improved ultrasound probes that provide for electromagnetic shielding.

Ultrasound transducers for medical applications have to comply with specific regulations that ensure protection against excessive temperatures and risk of cavitation. For example, the temperature of the patient contact surface should not exceed 43° C. under well defined test conditions. Mechanical Index is used as well by regulatories to quantify transducer performance as well as the maximum level according to the applications and imaging modes involved.

In practice, piezoelectric transducers are often thermally limited which means that the maximum temperature is reached without having reached the maximum mechanical index. This implies that the transducer can not be set to transmit the maximum acoustic pressure allowed by regulations, which can have a negative impact on image and Doppler signal quality.

Thus, it is desirable to implement solutions that contribute to reduce the front temperature of the transducer.

### BRIEF SUMMARY OF THE INVENTION

Embodiments of the present technology provide an ultrasound probe comprising: an acoustic stack including an active layer, a protection face plate or lens, and a matching layer disposed between the active layer and the protection face plate or lens, the matching layer comprising a mass layer including a first material and a spring layer including a second material that is different than the first material; and an electromagnetic radiation shield comprising the mass layer, the electromagnetic radiation shield encompassing the active layer, the electromagnetic radiation shield configured to inhibit external electromagnetic radiation from interfering with the signals communicated to and from the ultrasound probe via the cable.

In certain embodiments, the mass layer is thermally connected to a thermal drain or a heat sink such that heat is transferred from the mass layer to the thermal drain or heat sink and away from the protection face plate or lens.

In certain embodiments, the matching layer abuts the active layer or a ground electrode.

In certain embodiments, the matching layer abuts the protection face plate or lens.

In certain embodiments, the mass layer comprises a metal.

In certain embodiments, the spring layer comprises a polymer.

In certain embodiments, the spring layer comprises a material with an acoustic impedance of less than about 1.5 MegaRayls.

In certain embodiments, the mass layer has an impedance at least about five times greater than the spring layer.

In certain embodiments, the ultrasound probe further includes a cable configured to communicate signals to and from the ultrasound probe, wherein the electromagnetic radiation shield encompasses the cable, and wherein the electromagnetic radiation shield is grounded via an electrode.

In certain embodiments, the ultrasound probe is configured to communicate wirelessly with an ultrasound system.

Embodiments of the present technology provide a method of forming an ultrasound probe comprising: providing an acoustic stack including an active layer, a protection face plate or lens, and a matching layer disposed between the active layer and the protection face plate or lens, the matching layer comprising a mass layer including a first material and a spring layer including a second material that is different than the first material; and providing an electromagnetic radiation shield to the ultrasound probe, the electromagnetic radiation shield comprising the mass layer, the electromagnetic radiation shield encompassing the active layer, the electromagnetic radiation shield configured to inhibit external electromagnetic radiation from interfering with the signals communicated to and from the ultrasound probe.

Certain embodiments further include thermally connecting the mass layer to a thermal drain or heat sink such that heat is transferred from the mass layer to the thermal drain or heat sink and away from the protection face plate or lens.

Certain embodiments further include abutting the matching layer with the active layer or a ground electrode.

Certain embodiments further include abutting the matching layer with the protection face plate or lens.

In certain embodiments, the mass layer comprises a metal.

In certain embodiments, the spring layer comprises a polymer.

In certain embodiments, the spring layer comprises a material with an acoustic impedance of less than about 1.5 MegaRayls.

In certain embodiments, the mass layer has an impedance at least about five times greater than the spring layer.

Certain embodiments further include operably connecting a cable to the active layer, the cable configured to communicate signals to and from the ultrasound probe, wherein the electromagnetic radiation shield encompasses the cable, and wherein the electromagnetic radiation shield is grounded via an electrode.

In certain embodiments, the ultrasound probe is configured to communicate wirelessly with an ultrasound system.

### BRIEF DESCRIPTION OF THE DRAWINGS

FIG. 1 illustrates an ultrasound system formed in accordance with an embodiment of the present invention.

FIG. 2 illustrates a three-dimensional (3D) capable miniaturized ultrasound system formed in accordance with an embodiment of the present invention.

FIG. 3 illustrates a mobile ultrasound imaging system formed in accordance with an embodiment of the present invention.

FIG. 4 illustrates a hand carried or pocket-sized ultrasound imaging system formed in accordance with an embodiment of the present invention.

FIG. 5A illustrates components of an ultrasound system in accordance with an embodiment of the present invention.

FIG. 5B illustrates a matching section of the ultrasound transducer depicted in FIG. 5A.

FIG. 5C illustrates an alternative matching section of an ultrasound transducer in accordance with an embodiment of the present invention.

FIG. 5D illustrates components of an ultrasound system in accordance with an embodiment of the present invention.

FIGS. 6-9 illustrate acoustical simulations of the bandwidth performance of ultrasound probes in accordance with an embodiment of the present invention.

FIG. 10 illustrates a method of forming an ultrasound probe in accordance with an embodiment of the present invention.

### DETAILED DESCRIPTION OF CERTAIN EMBODIMENTS

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

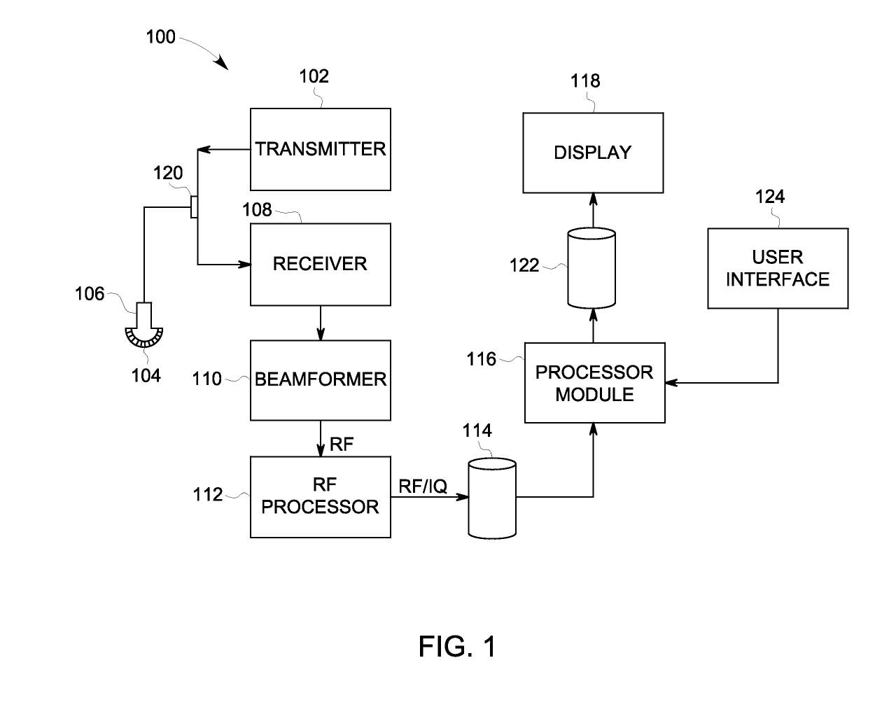

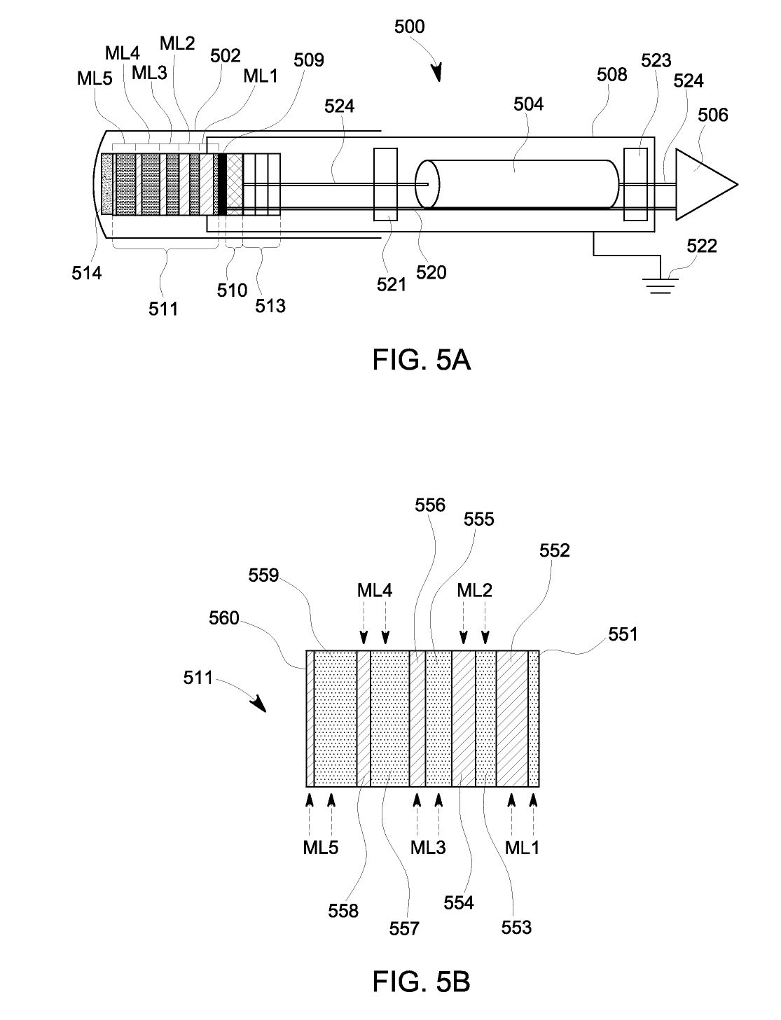

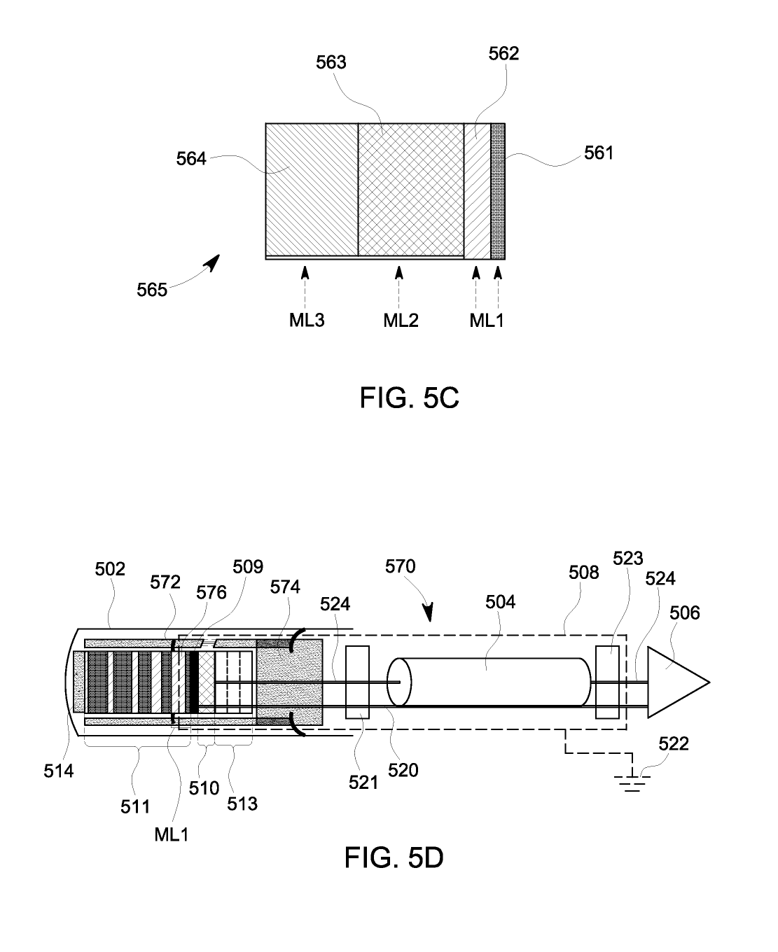

FIG. 1 illustrates an ultrasound system 100 including a transmitter 102 that drives an array of elements 104 (e.g., piezoelectric elements) within a probe 106 to emit pulsed ultrasonic signals into a body. The probe 106 may be configured as depicted in FIG. 5. The elements 104 may be arranged, for example, in one or two dimensions. A variety of geometries may be used. The system 100 may have a probe port 120 for receiving the probe 106 or the probe 106 may be hardwired to the system 100.

The ultrasonic signals are back-scattered from structures in the body, like fatty tissue or muscular tissue, to produce echoes that return to the elements 104. The echoes are received by a receiver 108. The received echoes are passed through a beamformer 110 that performs beamforming and outputs a radiofrequency (RF) signal. The RF signal then passes through an RF processor 112. Alternatively, the RF processor 112 may include a complex demodulator (not shown) that demodulates the RF signal to form in-phase and quadrature (IQ) data pairs representative of the echo signals. The RF or IQ signal data may then be routed directly to a memory 114 for storage.

The ultrasound system 100 also includes a processor module 116 to process the acquired ultrasound information (e.g., RF signal data or IQ data pairs) and prepare frames of ultrasound information for display on display 118. The processor module 116 is adapted to perform one or more processing operations according to a plurality of selectable ultrasound modalities on the acquired ultrasound information. Acquired ultrasound information may be processed and displayed in real-time during a scanning session as the echo signals are received. Additionally or alternatively, the ultrasound information may be stored temporarily in memory 114 or memory 122 during a scanning session and then processed and displayed in an off-line operation.

A user interface 124 may be used to input data to the system 100, adjust settings, and control the operation of the processor module 116. The user interface 124 may have a keyboard, trackball and/or mouse, and a number of knobs, switches or other input devices such as a touchscreen. The display 118 includes one or more monitors that present patient information, including diagnostic ultrasound images to the user for diagnosis and analysis. One or both of memory 114 and memory 122 may store two-dimensional (2D) and/or three-dimensional (3D) datasets of the ultrasound data, where such datasets are accessed to present 2D and/or 3D images. Multiple consecutive 3D datasets may also be acquired and stored over time, such as to provide real-time 3D or four-dimensional (4D) display. The images may be modified and the display settings of the display 118 also manually adjusted using the user interface 124.

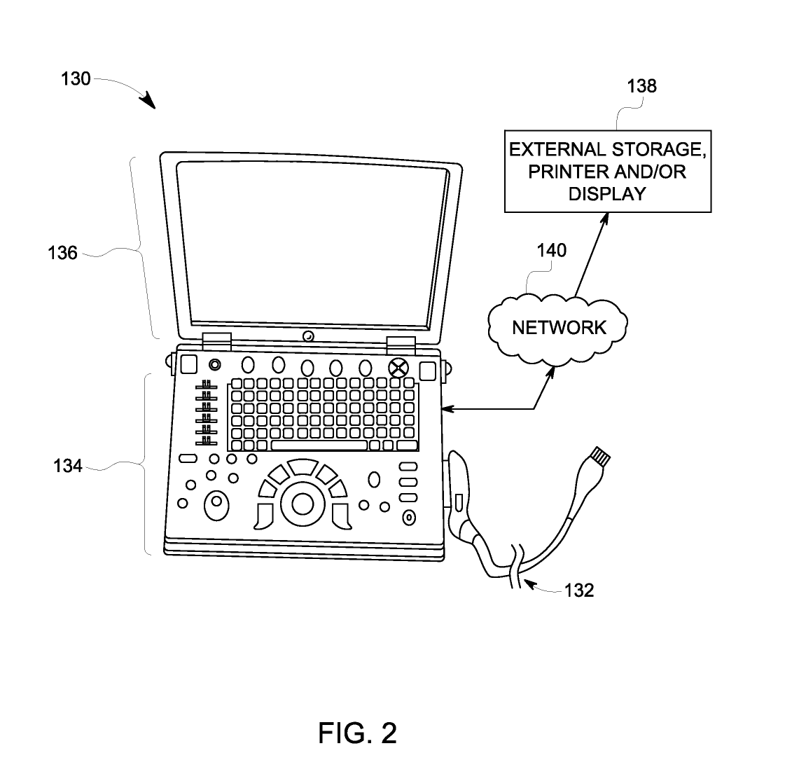

FIG. 2 illustrates a 3D-capable miniaturized ultrasound system 130 having a probe 132 that may include the probe configuration depicted in FIG. 5. The probe 132 may be configured to acquire 3D ultrasonic data. For example, the probe 132 may have a 2D array of transducer elements 104 as discussed previously with respect to the probe 106 of FIG. 1. A user interface 134 (that may also include an integrated display 136) is provided to receive commands from an operator.

As used herein, “miniaturized” means that the ultrasound system 130 is a handheld or hand-carried device or is configured to be carried in a person's hand, pocket, briefcase-sized case, or backpack. For example, the ultrasound system 130 may be a hand-carried device having a size of a typical laptop computer, for instance, having dimensions of approximately 2.5 inches in depth, approximately 14 inches in width, and approximately 12 inches in height. The ultrasound system 130 may weigh about ten pounds, and thus is easily portable by the operator. The integrated display 136 (e.g., an internal display) is also provided and is configured to display a medical image.

The ultrasonic data may be sent to an external device 138 via a wired or wireless network 140 (or direct connection, for example, via a serial or parallel cable or USB port). In some embodiments, external device 138 may be a computer or a workstation having a display. Alternatively, external device 138 may be a separate external display or a printer capable of receiving image data from the hand carried ultrasound system 130 and of displaying or printing images that may have greater resolution than the integrated display 136. It should be noted that the various embodiments may be implemented in connection with a miniaturized ultrasound system having different dimensions, weights, and power consumption.

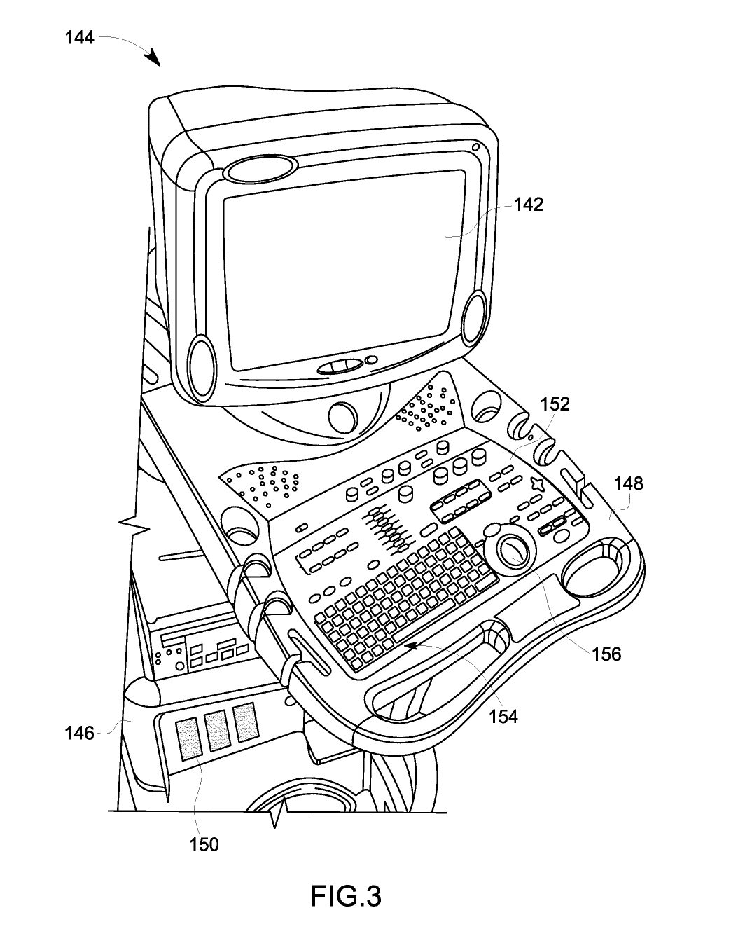

FIG. 3 illustrates a mobile ultrasound imaging system 144 provided on a movable base 146. The ultrasound imaging system 144 may also be referred to as a cart-based system. A display 142 and user interface 148 are provided and it should be understood that the display 142 may be separate or separable from the user interface 148. The system 144 has at least one probe port 150 for accepting probes (not shown) that may include the probe configuration depicted in FIG. 5.

The user interface 148 may optionally be a touchscreen, allowing the operator to select options by touching displayed graphics, icons, and the like. The user interface 148 also includes control buttons 152 that may be used to control the ultrasound imaging system 144 as desired or needed, and/or as typically provided. The user interface 148 provides multiple interface options that the user may physically manipulate to interact with ultrasound data and other data that may be displayed, as well as to input information and set and change scanning parameters. The interface options may be used for specific inputs, programmable inputs, contextual inputs, and the like. For example, a keyboard 154 and track ball 156 may be provided.

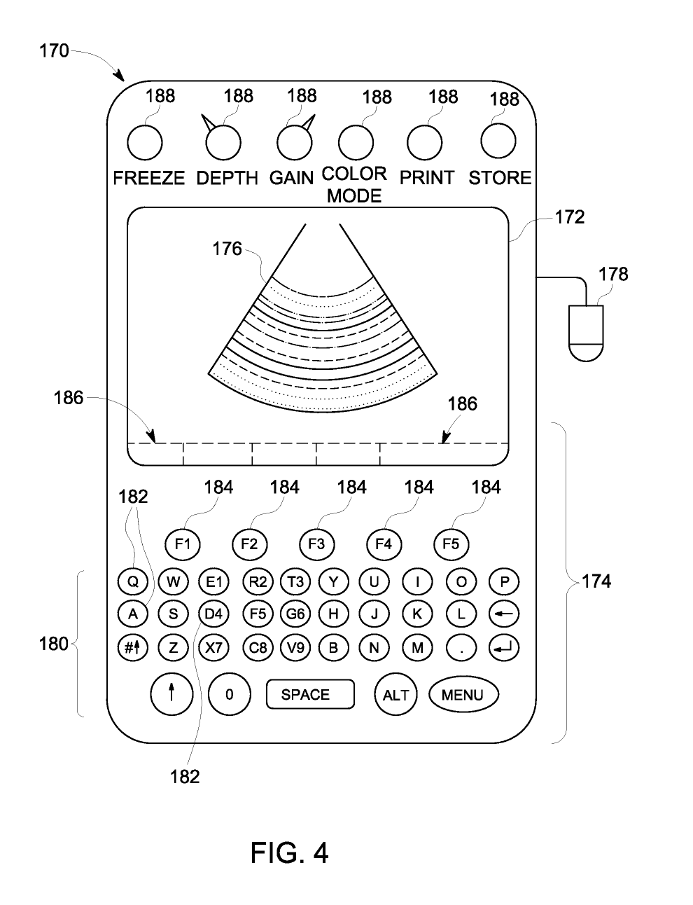

FIG. 4 illustrates a hand carried or pocket-sized ultrasound imaging system 170 wherein display 172 and user interface 174 form a single unit. By way of example, the pocket-sized ultrasound imaging system 170 may be approximately 2 inches wide, approximately 4 inches in length, and approximately 0.5 inches in depth and weighs less than 3 ounces. The display 172 may be, for example, a 320×320 pixel color LCD display (on which a medical image 176 may be displayed). A typewriter-like keyboard 180 of buttons 182 may optionally be included in the user interface 174. A probe 178 that may include the matching layer structure is interconnected with the system 170.

Multi-function controls 184 may each be assigned functions in accordance with the mode of system operation. Therefore, each of the multi-function controls 184 may be configured to provide a plurality of different actions. Label display areas 186 associated with the multi-function controls 184 may be included as necessary on the display 172. The system 170 may also have additional keys and/or controls 188 for special purpose functions, which may include, but are not limited to “freeze,” “depth control,” “gain control,” “color-mode,” “print,” and “store.”

FIG. 5A illustrates components 500 of an ultrasound system in accordance with an embodiment of the present invention. The components 500 include: an ultrasound probe 502, a cable 504, an amplifier 506, and an electromagnetic radiation shield 508. Probe 502 is configured to receive ultrasonic waves and convert them into electrical signals using electronic circuit 521 (e.g. flex circuit). Probe 502 communicates the electrical signals via cable 504 and electronic circuit 523 to amplifier 506. Amplifier 506 can be part of a receiver, such as the receiver 108 depicted in FIG. 1, for example.

Electromagnetic radiation shield 508 is incorporated into probe 502 such that it encapsulates active layer 510 of probe 502 and encapsulates the electrical signals communicated via cable 504 and electronic circuits 521, 523. Probe 502 includes a back-stack 513, active layer 510, ground electrode 509, matching section 511, and a protection face plate or lens 514. In Probe 502, active layer 510 includes at least one electroded piezoelectric material, either monolithic or piezocomposite, for example. In Probe 502, back-stack 513 can include any passive layers used to connect electrodes to the electrical circuit 521 and materials used to absorb the energy going to the back side of the transducer. In Probe 502, matching section 511 includes a plurality of matching layers ML1, ML2, ML3, ML4 and ML5, each matching layer including a spring layer comprising a spring material and a mass layer comprising a mass material. As depicted in FIG. 5B, the first matching layer ML1 includes spring layer 551 and mass layer 552; the second matching layer ML2 includes spring layer 553 and mass layer 554; the third matching layer ML3 includes spring layer 555 and mass layer 556; the fourth matching layer ML4 includes spring layer 557 and mass layer 558; the fifth matching layer ML5 includes spring layer 559 and mass layer 560. It should be understood that other designs may use a different number of matching layers and that all matching layers are not necessarily made of a spring layer and a mass layer. However, in certain embodiments at least one matching layer includes a spring layer and a mass layer, for example, as depicted in FIG. 5C, which depicts an alternative matching section 565 where the first matching layer ML1 includes spring layer 561 and mass layer 562, and the second and third matching layers ML2 563 and ML3 564, respectively, do not include both a spring layer and a mass layer.

As used herein, a spring material is a relatively low loss and low density material, such as a polymer or film, such as SU8™, an epoxy-based negative photoresist, or Kapton™, a polyimide material, a silicone and may have an acoustic impedance lower than 3 MegaRayls (MR) or even lower than 1.5 MR depending on the targeted acoustic impedance of the spring/mass matching layer. As used herein, a mass material is a relatively high density material such as tungsten, copper or other metal, and may have an acoustic impedance closer to 30 MR. It should be understood that other materials may be used.

In certain embodiments, each of the matching layer sections made of a spring layer and a mass layer has a thickness that is much less than quarter-wavelength, e.g. approximately 50 micrometers (μm), although other thicknesses are contemplated. In certain embodiments, the spring layer and the mass layer can each have a thickness of less than about ⅙ wavelength at center frequency, and the mass layer can have an impedance at least about five times greater than the spring layer. In certain embodiments, matching layers can have decreasing overall impedance such that the matching layer furthest from active layer 510 and closest to protection face plate or lens 514 has the lowest impedance of the matching layers. As depicted in FIG. 5B, decreasing overall impedance can be achieved in the matching layers by decreasing the thickness of the mass layer and increasing the thickness of the spring layer. For example, the matching layer with the highest impedance can have the highest percentage of mass material and the lowest percentage of spring material, and the matching layer with the lowest impedance can have the lowest percentage of mass material and the highest percentage of spring material.

As depicted, electromagnetic radiation shield 508 includes the mass layer of matching layer ML1. In other embodiments, electromagnetic radiation shield 508 can include a mass layer from any matching layer that includes both a mass layer and a spring layer. In certain embodiments, the mass layer that is incorporated into the electromagnetic radiation shield can be about 10μ thick or thicker. In certain embodiments, electromagnetic radiation shield 508 can include a mass layer from a matching layer that abuts active layer 510 and/or ground electrode 509. In certain embodiments, electromagnetic radiation shield 508 can include a mass layer from a matching layer that abuts protection face plate or lens 514. In certain embodiments, electromagnetic radiation shield 508 can include a mass layer from a matching layer that does not abut active layer 510, ground electrode 509 or protection face plate or lens 514.

Electromagnetic radiation shield 508 is grounded via electrode 520 to ground 522. In certain embodiments, electromagnetic radiation shield 508 can be grounded to a chassis console of an ultrasound system, for example. Active layer 510 of probe 502 is operably connected to amplifier 506 via electrode 524.

In certain embodiments, the ultrasound probe can be a wireless ultrasound probe that communicates with the ultrasound system without using a cable. In such embodiments, the electromagnetic radiation shield can include a mass layer of a matching layer of the probe and can encompass the active layer of the probe and the electronic circuit (e.g., flex circuit) of the probe. Such embodiments, can include a thermal drain/heat sink as discussed below, for example, in connection with FIG. 5D.

In operation, electromagnetic radiation shield 508 can prevent electromagnetic radiation generated by surrounding equipment from interfering with the electrical signals generated by active layer 510 and communicated between cable 504 and amplifier 506. Further, utilizing a mass layer of a matching layer as part of the electromagnetic radiation shield can allow the shield to be implemented without the undesirable effects usually associated with introducing a metal layer into an acoustical stack of an ultrasound transducer.

A technical effect of at least one embodiment is that the electromagnetic radiation shield prevents electromagnetic radiation generated by surrounding equipment from interfering with the electrical signals generated by a active layer of an ultrasound transducer and communicated between a cable and an amplifier of a receiver.

FIG. 5D illustrates components of an ultrasound system 570 in accordance with an embodiment of the present invention. The ultrasound system 570 includes the components described above in connection with FIG. 5A, and further includes a thermal drain/heat sink 574 disposed adjacent to back-stack 513. As depicted in FIG. 5D, thermal drain/heat sink 574 is thermally connected to mass layer 576 in the first matching layer ML1 via thermal connection 572, which is configured to transfer heat from mass layer 576 to thermal drain/heat sink 574 and away from protection face plate or lens 514. In certain embodiments, thermal drain/heat sink 574 can be thermally connected to any and/or all mass layers in the matching section via a thermal connection configured to transfer heat from the mass layer(s) to thermal drain/heat sink 574. In certain embodiments, thermal drain/heat sink 574 can be part of electromagnetic radiation shield 508 and can be connected to ground 522 via electrode 520.

A technical effect of at least one embodiment is that heat from one or more mass layers in a matching section of an ultrasound probe is directed away from the protection face plate or lens toward a thermal drain/heat sink at the back-end of the transducer, thereby reducing the operating temperature of the protection face plate or lens.

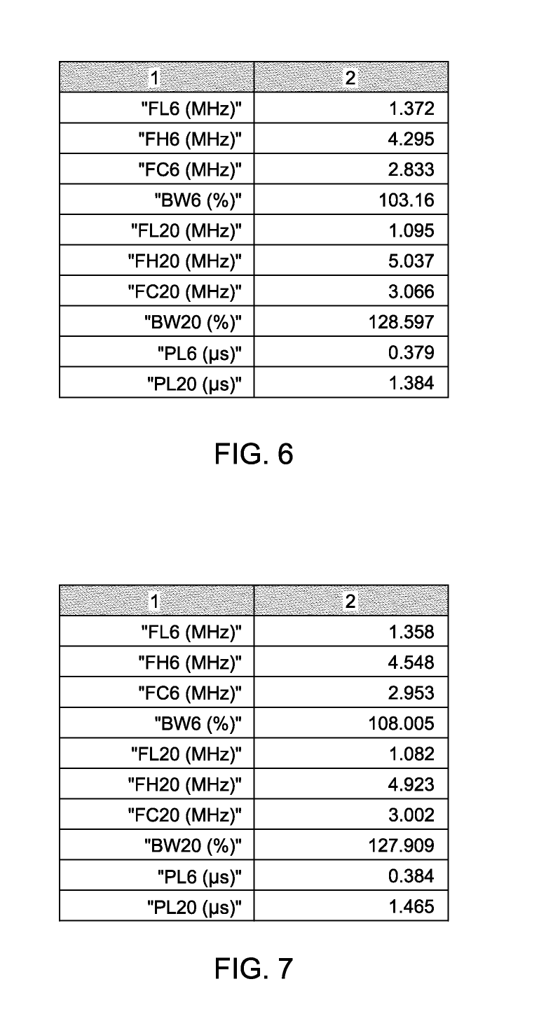

FIGS. 6-9 illustrate acoustical simulations of the bandwidth performance of ultrasound probes in accordance with an embodiment of the present invention where outer matching layer is a mass-spring structure. FIG. 6 depicts bandwidth performance for a prototype ultrasound probe with an inventive electromagnetic radiation shield, and the following acoustic stack properties: an active stack comprising a piezoelectric layer made of composite PZT with a thickness designed in such way that the resonance frequency of the active stack in air without any matching layers nor lens is about 3 MHz i.e. close to the desired center frequency for the probe a matching layer made of a material which has about 11.5MRay acoustic impedance with a thickness of 200 μm, a matching layer made of a material which has about 5MRay acoustic impedance with a thickness of 170 μm, and a matching layer including a spring layer made of silicone with a thickness of 25 μm undiced and a mass layer made of Copper with a thickness of 7 μm undiced. The transducer was operated at 220 μm pitch 40 μm kerf.

FIG. 7 depicts bandwidth performance for an ultrasound probe without an inventive electromagnetic radiation shield, and the following acoustic stack properties: active layer made of composite PZT with a thickness designed in such way that the resonance frequency of the active stack in air without any matching layers nor lens is about 3 MHz, a matching layer made of a material which has about 11.5MRay acoustic impedance with a thickness of 200 μm, a matching layer made of a material which has about 5MRay acoustic impedance with a thickness of 170 μm, and a matching layer made of a material which has about 2 MRay acoustic impedance with a thickness of 140 μm undiced. The transducer was operated at 220 μm pitch 40 μm kerf.

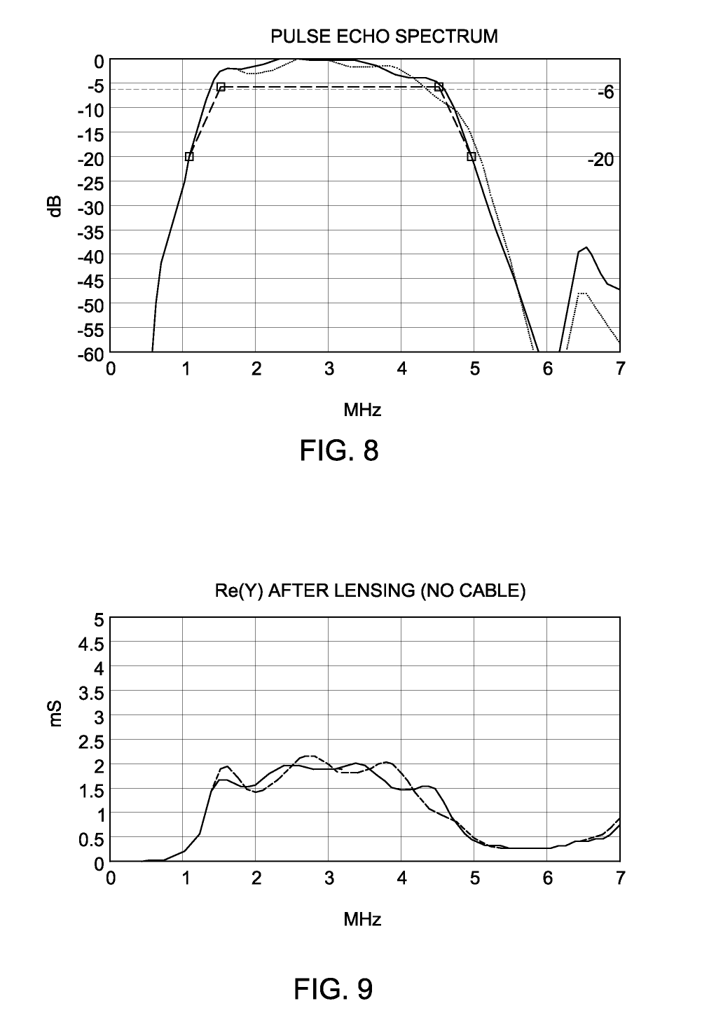

FIGS. 8-9 graph the acoustic performance of the prototype ultrasound probe with an inventive electromagnetic radiation shield and the ultrasound probe without an inventive electromagnetic radiation shield. Notably, the results observed and provided in FIGS. 6-9 are similar, which indicates that the inventive electromagnetic radiation shield did not detract from acoustic performance.

Methods of making and using an ultrasound probe with an inventive electromagnetic radiation shield as described above are also contemplated and are considered to be part of the present invention.

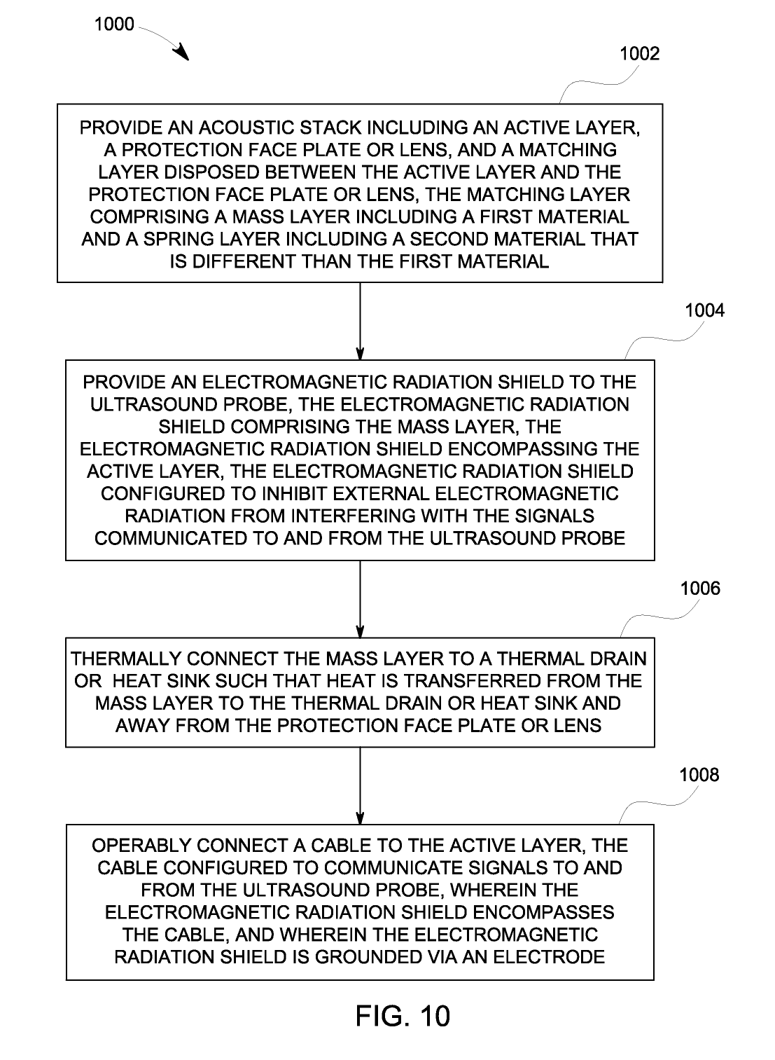

FIG. 10 illustrates a method 1000 of forming an ultrasound probe in accordance with an embodiment of the present invention. At 1002, an acoustic stack is provided that includes a active layer, a protection face plate or lens, and a matching layer disposed between the active layer and the protection face plate or lens, the matching layer comprising a mass layer including a first material and a spring layer including a second material that is different than the first material. At 1004, an electromagnetic radiation shield is provided to the ultrasound probe, the electromagnetic radiation shield comprising the mass layer, the electromagnetic radiation shield encompassing the active layer, the electromagnetic radiation shield configured to inhibit external electromagnetic radiation from interfering with the signals communicated to and from the ultrasound probe via the cable. At 1006, the mass layer is thermally connected to a thermal drain or heat sink such that heat is transferred from the mass layer to the thermal drain or heat sink and away from the protection face plate or lens. At 1008, a cable is operably connected to the active layer, e.g., using a flexible circuit, the cable configured to communicate signals to and from the ultrasound probe, wherein the electromagnetic radiation shield encompasses the cable, and wherein the electromagnetic radiation shield is grounded via an electrode.

In certain embodiments, the method can further include abutting the matching layer with the active layer and/or a ground electrode. In certain embodiments, the method can further include abutting the matching layer with the protection face plate or lens. In certain embodiments, the mass layer comprises a metal. In certain embodiments, the spring layer comprises a polymer. In certain embodiments, the spring layer comprises a material with an acoustic impedance of less than about ⅕ piezoelectric material impedance. In certain embodiments, the mass layer has impedance at least about five times greater than the spring layer.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An ultrasound probe comprising:
an acoustic stack including an active layer, a protection face plate or lens, and a matching layer disposed between the active layer and the protection face plate or lens, the matching layer comprising a mass layer including a first material and a spring layer including a second material that is different than the first material; and
an electromagnetic radiation shield comprising the mass layer, the electromagnetic radiation shield encompassing the active layer, the electromagnetic radiation shield configured to inhibit external electromagnetic radiation from interfering with the signals communicated to and from the ultrasound probe.

2. The ultrasound probe of claim 1, wherein the mass layer is thermally connected to a thermal drain or a heat sink such that heat is transferred from the mass layer to the thermal drain or heat sink and away from the protection face plate or lens.

3. The ultrasound probe of claim 1, wherein the matching layer abuts the active layer or a ground electrode.

4. The ultrasound probe of claim 1, wherein the matching layer abuts the protection face plate or lens.

5. The ultrasound probe of claim 1, wherein the mass layer comprises a metal.

6. The ultrasound probe of claim 1, wherein the spring layer comprises a polymer.

7. The ultrasound probe of claim 1, wherein the spring layer comprises a material with an acoustic impedance of less than about 1.5 MegaRayls.

8. The ultrasound probe of claim 1, wherein the mass layer has an impedance at least about five times greater than the spring layer.

9. The ultrasound probe of claim 1, further including a cable configured to communicate signals to and from the ultrasound probe, wherein the electromagnetic radiation shield encompasses the cable, and wherein the electromagnetic radiation shield is grounded via an electrode.

10. The ultrasound probe of claim 1, wherein the ultrasound probe is configured to communicate wirelessly with an ultrasound system.

11. A method of forming an ultrasound probe comprising:
providing an acoustic stack including an active layer, a protection face plate or lens, and a matching layer disposed between the active layer and the protection face plate or lens, the matching layer comprising a mass layer including a first material and a spring layer including a second material that is different than the first material; and
providing an electromagnetic radiation shield to the ultrasound probe, the electromagnetic radiation shield comprising the mass layer, the electromagnetic radiation shield encompassing the active layer, the electromagnetic radiation shield configured to inhibit external electromagnetic radiation from interfering with the signals communicated to and from the ultrasound probe via the cable.

12. The method of claim 11, further including thermally connecting the mass layer to a thermal drain or heat sink such that heat is transferred from the mass layer to the thermal drain or heat sink and away from the protection face plate or lens.

13. The method of claim 11, further including abutting the matching layer with the active layer or a ground electrode.

14. The method of claim 11, further including abutting the matching layer with the protection face plate or lens.

15. The method of claim 11, wherein the mass layer comprises a metal.

16. The method of claim 11, wherein the spring layer comprises a polymer.

17. The method of claim 11, wherein the spring layer comprises a material with an acoustic impedance of less than about 1.5 MegaRayls.

18. The method of claim 11, wherein the mass layer has an impedance at least about five times greater than the spring layer.

19. The method of claim 11, further including operably connecting a cable to the active layer, the cable configured to communicate signals to and from the ultrasound probe, wherein the electromagnetic radiation shield encompasses the cable, and wherein the electromagnetic radiation shield is grounded via an electrode.

20. The method of claim 11, wherein the ultrasound probe is configured to communicate wirelessly with an ultrasound system.
